(19) United States
(12) Patent Application Publication
(10) Pub. No.: US 2021/0290728 A1
(43) Pub. Date: Sep. 23, 2021

(54) Use of ST266 to Treat Severe Systemic Inflammation and Post-Acute COVID-19 Syndrome

(71) Applicant: Noveome Biotherapeutics, Inc., Pittsburgh, PA (US)

(72) Inventor: Larry R Brown, Newton, MA (US)

(73) Assignee: Noveome Biotherapeutics, Inc., Pittsburgh, PA (US)

(21) Appl. No.: 17/197,580

(22) Filed: Mar. 10, 2021

Related U.S. Application Data
(60) Provisional application No. 62/991,962, filed on Mar. 19, 2020.
(60) Provisional application No. 63/064,495, filed on Aug. 12, 2020.

(51) Int. Cl. A61K 38/18; A61K 38/19; A61P 29/00

(52) U.S. Cl. CPC A61K 38/18; A61P 29/00; A61K 38/19

(57) ABSTRACT

The invention is directed to methods for the treatment of severe systemic inflammatory responses, including but not limited to the severe systemic inflammatory response called a “cytokine storm”. The invention is further directed to the use of ST266 to treat severe systemic inflammatory responses. Specifically, the invention is directed to methods for treating a cytokine storm or sequelae thereof by intravenously or intranasally administering ST266 to a subject suffering from such symptoms.

### FIELD OF THE INVENTION

The field of the invention is directed to methods for the treatment of severe systemic inflammatory responses, including but not limited to the severe systemic inflammatory response called a “cytokine storm”. The field of the invention is further directed to the use of ST266 to treat severe systemic inflammatory responses. Specifically, the field of the invention is directed to methods for treating a cytokine storm or sequelae thereof by intravenously or intranasally administering ST266 to a subject suffering from such symptoms.

### BACKGROUND OF THE INVENTION

There are number of things that can trigger the severe systemic inflammatory responses that are characterized by a cytokine storm. Systemic Inflammatory Response Syndrome (SIRS) is an umbrella term referring to an extreme defense response of the body due to a stressor of some kind, including infection (then referred to as sepsis), trauma, surgery, drug overdose, etc. A diagnosis of SIRS is determined by the occurrence of any two (or more) of the following criteria: Body temperature over 38 or under 36 degrees Celsius, resting heart rate greater than 90 beats/minute, resting respiratory rate greater than 20 breaths/minute or partial pressure of CO₂ less than 32 mmHg, and leucocyte count greater than 12000 or less than 4000/mL or over 10% immature forms or bands. However, not all patients that have severe systemic inflammatory responses exhibit the SIRS criteria, making diagnosis and treatment challenging. Regardless of the stressor triggering the severe response or the presence of all, some or none of the SIRS criteria, it is critical that the cytokine storm be controlled as soon as possible to prevent body-wide damage including multiple organ failure and death.

The coronavirus pandemic of 2020 has brought renewed attention to cytokine storms. A cytokine storm (also called Cytokine Release Syndrome (CRS) or hypercytokinemia) is essentially a condition wherein the body releases too many cytokines and other inflammatory substances into the blood too quickly as the result of an infection, an autoimmune condition, trauma, myocardial infarction, burns and numerous other triggers. It can be severe or life-threatening and often causes multiple organ failure. A cytokine storm can result in the release of more than 150 known inflammatory mediators (cytokines, oxygen free radicals, and coagulation factors). Tisoncik, J. R. et al. Microbiol. Mol. Biol. Rev. 2012 March, 76(1):16-32.

ST266 is a novel platform biologic that contains hundreds of bioactive molecules, many of which are anti-inflammatory, and all of which are at physiological concentrations (pg/mL to ng/mL). It is made under cGMP conditions by selecting a subpopulation of amnion epithelial cells and culturing them under proprietary conditions such that the cells secrete a novel secretome. The novel secretome is collected, tested for potency and consistency, and is the ST266 drug substance. More details can be found in U.S. Pat. Nos. 8,088,732, 8,278,095 and 8,741,646, each incorporated herein in their entirety. Briefly, ST266 is made by obtaining a placenta and isolating an amnion from the placenta, enzymatically releasing amnion-derived epithelial cells from the amnion, collecting the released amnion-derived epithelial cells, culturing the collected amnion-derived epithelial cells in basal culture medium that is supplemented with 0.5% human serum albumin and 5-10 ng/mL recombinant human EGF, removing the culture medium after about 2-3 days and applying fresh culture medium, and collecting the culture medium after culturing the cells for 2-3 days. The collection of the culture media after culture, the addition of fresh culture media, the culturing for 2-3 days, and collecting the culture media can be repeated a plurality of times.

Because ST266 is comprised of so many bioactive molecules, it impacts numerous pathways simultaneously using physiological concentrations of the molecules. This is fundamentally different from the traditional one drug-one target paradigm seen in traditional drug development where orders of magnitude larger doses are administered. Applicant believes, based on data presented below in the Examples, that ST266 is uniquely suited to treat the severe systemic inflammatory response seen in cytokine storm.

Despite the fact that the literature is replete with references to possible treatments for cytokine storm, currently, there are few satisfactory therapies. Corticosteroids are commonly used to suppress inflammation. Interferon-gamma (IFN-γ) primarily activates epithelial cells and reduces the mononuclear macrophage-mediated proinflammatory activity of IFN-αβ (Davidson, et al. Science, 10 Jun. 2020, 369(6504):712-717). IFNλ is a potent anti-influenza therapeutic without the inflammatory side effects of IFNα treatment. (EMBO Molecul Med. 2016; 8(9):1099-1112. PubMed PMID: 27520969). IL-1 family antagonists, IL-6 family antagonists, TNF blockers, among others, have also been considered as potential treatments.

It is believed that a treatment option that could down-regulate the severe systemic inflammatory response seen in cytokine storm would vastly benefit patients who otherwise have few treatment options. Applicant believes that systemically delivered ST266 and its anti-inflammatory activity is a unique treatment option.

In addition, a need exists for a treatment that lessens the likelihood or severity of long-term effects of COVID-19 infection, including the loss of sense of taste and smell, inflammatory conditions of the brain, or other conditions experienced by patients that have or even have recovered from COVID-19 such as “brain fog”. Targeted intranasal delivery of ST266 and its neuroprotective and anti-inflammatory activity is a unique treatment option. In addition, because COVID-19 infection may be a disease of the microvasculature (Circulation. 2020; 142:1609-1611), systemically delivered ST266 may ameliorate or lessen long-term effects of COVID-19 infection. The endothelial cell protective and anti-inflammatory effects ST266 are described in detail in the Examples below.

### BRIEF SUMMARY OF THE INVENTION

ST266 presents a unique opportunity to administer a multitargeted cytokine storm treatment to patients. ST266 is composed of multiple growth factors and anti-inflammatory cytokines (D. Steed et al., Eplasty. 8, 157-165 (2008). It is produced under cGMP standards using a novel culturing process for proprietary Amnion-derived Multipotent Progenitor (AMP) cells. The AMP cell's secretome is collected and is the ST266 product and the cells themselves are discarded. Thus, ST266 is essentially “cell therapy without the cell” but without the supply chain constraints and safety concerns associated with cell therapy such as mesenchymal stem cells (MSCs). ST266 has been shown to be safe and effective in numerous preclinical and clinical trials.

In two human clinical trials, topical oral administration of ST266 to the gums significantly reduced the inflammatory cytokines IL-1β, TNF-α and reduced IL-17α in gingival crevicular fluid. (NCT02761993). Another clinical trial of the inflammatory response to ultraviolet light showed that topical dermal administration of ST266 to the skin significantly reduced erythema. This study also showed elevated xeroderma pigmentosum group A protein (XPA) levels. XPA significantly influences expression of a subset of genes important for mitochondrial function which is critical to cell survival (Guan, L., et al. Clin. Cosmet. Investig. Dermatol. 10, 459-471 (2017)).

Other peer-reviewed published studies have shown that locally administered ST266 significantly reduces inflammatory cytokines. One study showed that intracranially administered ST266 delivered to a penetrating brain injury wound significantly reduced inflammatory neutrophil cell infiltration and promoted significant cellular and functional neuroprotection. (Deng-Bryant, Y. et al. Restor. Neurol. Neurosci. 33, 189-203 (2015), Deng-Bryant, Y. et al. J. Trauma Acute Care Surg. 73, S156-64 (2012)).

Additional preclinical studies have shown that intranasally delivered ST266 reduced inflammation in brain tissues and prevented cell death through multiple biochemical pathways. (Khan, R. S. et al. Sci. Rep. 7, 41768 (2017), Grinblat, G. A. et al. Investig. Ophthalmol. Vis. Sci. 59, 2470-2477 (2018), Khan, R. S., et al. J. Neuro-Ophthalmology (2019)).

In animal models of optic nerve disease and injury, ST266 was administered via the targeted intranasal route to the cribriform plate to allow diffusion via the olfactory nerves into the eye and brain. Targeted intranasal ST266 delivery reached the brain and optic nerve and was able to reverse vision loss in an animal model of optic neuritis (R. S. Khan et al., Sci. Rep. 7, 41768 (2017)) and reduce visual acuity loss in acute traumatic optic nerve crush (G. A. Grinblat et al., Investig. Ophthalmol. Vis. Sci. 59, 2470-2477 (2018)). These studies demonstrated clear neuroprotection, anti-inflammation, prevention of demyelination and increased retinal ganglion cell numbers.

Despite the successful demonstration of the anti-inflammatory properties of ST266 delivered topically, intracranially or intranasally, its anti-inflammatory capabilities and usefulness to treat cytokine storm when delivered systemically has, heretofore, not been demonstrated. This is due, in part, to the fact that ST266 contains physiological levels of hundreds of cytokines, growth factors, and other therapeutic components that would be expected to be too diluted upon systemic injection to be active. Yet, as described herein, and for the first time, Applicant has demonstrated that systemically (intravenous, parenteral) administered ST266 provides a potent therapy for down-regulating the severe inflammatory response that characterizes a cytokine storm.

Applicant's novel approach to targeted intranasal delivery of ST266 enables its use for COVID-19 or SIRS-related inflammatory conditions of the brain.

Accordingly, a first aspect of the invention is a method for treating a severe systemic inflammatory response, including cytokine storm, in a patient in need thereof comprising the step of systemically administering to the patient a therapeutically effective dose of ST266.

A specific embodiment of aspect one is wherein the severe systemic inflammatory response, including cytokine storm, is induced by an infectious or non-infectious stressor.

Another specific embodiment of aspect one is wherein the infectious stressor is selected from the group consisting of bacteria, viruses, and yeasts.

Another specific embodiment of aspect one is wherein the virus is a coronavirus.

Another specific embodiment of aspect one is wherein the coronavirus is SARS-CoV-2.

Another specific embodiment of aspect one is wherein the non-infectious stressor is selected from the groups consisting of surgery, trauma, autoimmune disease, burns, myocardial infarction, and chimeric antigen receptor (CAR)-T cell therapy.

Another specific embodiment of aspect one is wherein the systemic administration is intravenous administration. Another specific embodiment of aspect one is wherein the systemic administration is parenteral administration.

Another specific embodiment of aspect one is wherein the therapeutically effective dose of ST266 is 0.01 mL/kg to 100 mL/kg. Another embodiment is wherein the range of ST266 is 0.5 to 1.0 mL/kg once or twice a day.

Another specific embodiment of aspect one is wherein the severe inflammatory response is Systemic Inflammatory Response Syndrome (SIRS), including that seen in sepsis.

Another specific embodiment of aspect one is a method for treating cytokine storm induced by COVID-19 infection in a patient in need thereof comprising the step of administering to a patient a therapeutically effective dose of ST266, wherein the administration is intravenous administration and the therapeutically effective dose is 0.01 mL/kg to 100 mL/kg.

A second aspect of the invention is a method of treating long-term effects of COVID-19 infection, including loss of sense of taste (ageusia) and smell (anosmia), inflammatory conditions of the brain, brain fog, or other conditions experienced by patients that have or have recovered from COVID-19 infection comprising intranasal delivery of ST266.

Aspect three of the invention is a ST266 composition for use in a method for treating a severe systemic inflammatory response in a patient in need thereof, wherein the ST266 is administered intravenously to the patient.

In one embodiment, the ST266 composition for use in aspect three of the invention is wherein the severe systemic inflammatory response is a cytokine storm.

Aspect four of the invention is a ST266 composition for use in a method for treating post-acute COVID-19 syndrome in a patient in need thereof, wherein the ST266 is administered by targeted intranasal delivery to the patient.

In one embodiment, the ST266 composition for use in aspect four of the invention is wherein the post-acute COVID-19 syndrome is characterized by symptoms selected from the group consisting of anosmia, ageusia, neuro-inflammation, and brain fog.

In one embodiment, the ST266 composition for use in aspects three and four of the invention is wherein the ST266 composition comprises cytokines VEGF, TGFβ2, Angiogenin, PDGF and the MMP inhibitors TIMP-1 and/or TIMP-2.

In another embodiment, the ST266 composition for use in aspects three and four is wherein the physiological range of the cytokines is ˜5-16 ng/mL for VEGF, ˜2.5-2.7 ng/mL for TGFβ2, ˜3.5-4.5 ng/mL for Angiogenin, ˜100-165 pg/mL for PDGF, ˜0.68 μg/mL for TIMP-1, and ˜1.04 μg/mL for TIMP-2.

### FURTHER EMBODIMENTS OF THE INVENTION

Embodiment A: A pharmaceutical composition comprising ST266 for use in treating a severe systemic inflammatory response in a patient in need thereof by systemic administration to the patient of a therapeutically effective dose of ST266.

Embodiment B: The pharmaceutical composition for use according to Embodiment A, wherein the severe systemic inflammatory response is induced by an infectious or non-infectious stressor.

Embodiment C: The pharmaceutical composition for use according to Embodiment B, wherein the infectious stressor is selected from the group consisting of bacteria, viruses, and yeasts.

Embodiment D: The pharmaceutical composition for use according to Embodiment C, wherein the virus is a coronavirus.

Embodiment E: The pharmaceutical composition for use according to Embodiment D, wherein the coronavirus is SARS-CoV-2.

Embodiment F: The pharmaceutical composition for use according to any one of Embodiments B-E, wherein the non-infectious stressor is selected from the groups consisting of surgery, trauma, autoimmune disease, burns, myocardial infarction, and chimeric antigen receptor (CAR)-T cell therapy.

Embodiment G: The pharmaceutical composition for use according to any one of Embodiments A-F, wherein the systemic administration is intravenous administration.

Embodiment H: The pharmaceutical composition for use according to any one of Embodiments A-G, wherein the therapeutically effective dose of ST266 is 0.01 mL/kg to 100 mL/kg.

Embodiment I: The pharmaceutical composition for use according Embodiment H, wherein the therapeutically effective dose of ST266 is 0.1 mL/kg to 1 mL/kg.

Embodiment J: The pharmaceutical composition for use according to any one of Embodiments A-I, wherein the severe systemic inflammatory response is Systemic Inflammatory Response Syndrome (SIRS).

Embodiment K: The pharmaceutical composition for use according to any one of Embodiments A-I, wherein the severe systemic inflammatory response is cytokine storm.

Embodiment L: A pharmaceutical composition comprising ST266 for use in treating cytokine storm induced by COVID-19 infection in a patient in need thereof by administration to the patient of a therapeutically effective dose of ST266, wherein the administration is intravenous administration and the therapeutically effective dose is 0.01 mL/kg to 100 mL/kg.

Embodiment M: The pharmaceutical composition for use according to Embodiment L, wherein the dosage range of ST266 is 0.1 mL/kg to 1.0 mL/kg once a day or 0.1 mL/kg to 1.0 mL/kg twice a day.

Embodiment N: A pharmaceutical composition comprising ST266 for use in treating post-acute COVID-19 syndrome (PACS) in a patient in need thereof by targeted administration to the patient of a therapeutically effective dose of ST266.

Embodiment O: The pharmaceutical composition for use according to Embodiment N, wherein the therapeutically effective dose of ST266 is 100 μL/kg-1 mL/kg and the administration is systemic.

Embodiment P: The pharmaceutical composition for use according to Embodiment N, wherein the therapeutically effective dose of ST266 is 200 μL-400 μL daily and the administration is intranasal.

Embodiment Q: The pharmaceutical composition for use according to any one of Embodiments A-P, wherein ST266 comprises physiologic concentrations of VEGF, TGFβ2, Angiogenin, PDGF, TIMP-1 and TIMP-2, wherein the physiologic concentration is ˜5-16 ng/mL for VEGF, ˜2.5-2.5 ng/mL for TGFβ2, ˜3.5-4.5 ng/mL for Angiogenin, ˜100-165 pg/mL for PDGF, ˜0.68 μg/mL for TIMP-1 and ˜1.04 μg/mL for TIMP-2.

Embodiment R: The pharmaceutical composition for use according to any one of Embodiments A-Q, wherein ST266 is obtained or obtainable by the method of:
(a) isolating amnion epithelial cells from the amnion of a placenta;
(b) selecting Amnion-derived Multipotent Progenitor (AMP) cells from the amnion epithelial cells;
(c) culturing the AMP cells in animal-free basal culture medium, wherein the basal culture medium is supplemented with clinical grade human albumin added up to a concentration of 10% and 5-10 ng/mL EGF;
(d) removing the basal culture medium after about 2-3 days and applying fresh basal culture medium;
(e) culturing the cells in the basal culture medium of step (d) for about 2-3 days; and
(f) collecting the basal culture medium of step (e) to obtain ST266.

Embodiment S: The pharmaceutical composition for use according to Embodiment R, wherein one or more of steps (d)-(f) is repeated a plurality of times and ST266 obtained in step (f) is combined to create a pooled ST266.

Embodiment T: The pharmaceutical composition for use according to Embodiments R or S, wherein the basal culture medium is serum-free.

### Definitions

As defined herein “isolated” refers to material removed from its original environment and is thus altered “by the hand of man” from its natural state.

As used herein, the term “Amnion-derived Multipotent Progenitor cell” or “AMP cell” means a specific population of cells that are epithelial cells derived from the amnion of a placenta. AMP cells secrete a unique combination of physiologically relevant cytokines in a physiologically relevant temporal manner into the extracellular space or into surrounding culture media. AMP cells have not been cultured in the presence of any non-human animal-derived products, making them and cell products derived from them suitable for human clinical use. In another embodiment, the AMP cells secrete the cytokines VEGF, TGFβ2, Angiogenin, PDGF and the MMP inhibitors TIMP-1 and/or TIMP-2. The physiological range of the cytokine or cytokines in the unique combination is as follows: ˜5-16 ng/mL for VEGF, ˜2.5-2.7 ng/mL for TGFβ2-3.5-4.5 ng/mL for Angiogenin, ˜100-165 pg/mL for PDGF, ˜0.68 μg/mL for TIMP-1, and ˜1.04 μg/mL for TIMP-2. They grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion epithelial cells, from which AMP cells are selected, have no reaction with an antibody to the stem/progenitor cell marker c-kit (CD117), and minimal to no reaction with an antibody to the stem/progenitor cell marker Thy-1 (CD90).

By the term “animal-free” when referring to certain compositions, growth conditions, culture media, etc., described herein, is meant that no non-human animal-derived materials, such as bovine serum, proteins, lipids, carbohydrates, nucleic acids, vitamins, etc., are used in the preparation, growth, culturing, expansion, storage or formulation of AMP cells and their secreted product ST266, composition or process. By “no non-human animal-derived materials” is meant that the materials have never been in or in contact with a non-human animal body or substance, so they are not xeno-contaminated. Only clinical grade materials, such as recombinantly produced human proteins, are used in the preparation, growth, culturing, expansion, storage and/or formulation of AMP cells and ST266.

By the term “serum-free” is meant that no non-human animal-derived serum is used in the preparation, growth, culturing, expansion, storage or formulation of AMP cells and their secreted product ST266.

As used herein, “conditioned medium” is a medium in which a specific cell or population of cells has been cultured, and then the cells are removed from the medium. When cells are cultured in a medium, they secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium.

As used herein, the term “ST266” (previously termed “Amnion-derived Cellular Cytokine Solution” or “ACCS”) means conditioned medium that has been made by culturing AMP cells and contains the cytokines VEGF, TGFβ2, Angiogenin, PDGF and the MMP inhibitors TIMP-1 and/or TIMP-2. The physiological range of the cytokine or cytokines in the unique combination is as follows: ˜5-16 ng/mL for VEGF, ˜2.5-2.7 ng/mL for TGFβ2, ˜3.5-4.5 ng/mL for Angiogenin, ˜100-165 pg/mL for PDGF, ˜0.68 μg/mL for TIMP-1, and ˜1.04 μg/mL for TIMP-2.

The term “physiologic” or “physiological level” as used herein means the level that a substance in a living system is found and that is relevant to the proper functioning of a cellular, biochemical and/or biological process.

The term “cell product” or “cell products” as used herein refers to any and all substances made by and released or secreted from a cell, including but not limited to, protein factors (i.e., growth factors, differentiation factors, engraftment factors, cytokines, morphogens, proteases, extracellular matrix components, etc.

The term “therapeutically effective amount” means that amount of a therapeutic agent necessary to achieve a desired physiological effect (i.e., treating a severe systemic inflammatory response).

As used herein, the term “pharmaceutically acceptable” means that the components, in addition to the therapeutic agent, comprising the formulation, are suitable for administration to the patient being treated in accordance with the present invention.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

As used herein, the term “tissue” refers to an aggregation of similarly specialized cells united in the performance of a particular function.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

As used herein, the term “agent” means an active agent or an inactive agent. By the term “active agent” is meant an agent that is capable of having a physiological effect when administered to a subject. Non-limiting examples of active agents include growth factors, cytokines, antibiotics, cells, conditioned media from cells, etc. By the term “inactive agent” is meant an agent that does not have a physiological effect when administered. Such agents may alternatively be called “pharmaceutically acceptable excipients”. Non-limiting examples include time release capsules and the like.

The terms “parenteral administration” and “administered parenterally” are art-recognized and refer to modes of administration other than nasal, targeted intranasal, enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, epidural, intracerebral, intraocular, intracameral, subdural and intrasternal injection or infusion.

As used herein, the term “enteral” administration means any route of drug administration that involves absorption of the drug through the gastrointestinal tract. Enteral administration may be divided into three different categories: oral, gastric, and rectal.

As used herein, the term “topical” administration means a medication that is applied to body surfaces such as the skin, eye or mucous membranes to treat ailments using a large range of formulations including, but not limited to, liquids, sprays, creams, foams, gels, lotions, salves, powders and ointments.

The term “intranasal” or “intranasal delivery” or “intranasal administration” or “targeted intranasal” as used herein means delivery through the nasal cavity to the olfactory epithelium adjacent to the cribriform. Such administration utilizes a targeted intranasal delivery device.

The terms “sustained-release”, “extended-release”, “time-release”, “controlled-release”, or “continuous-release” as used herein means an agent, typically a therapeutic agent or drug, that is formulated to dissolve slowly and be released over time.

As used herein, the term “vascular permeability” means the capacity of a blood vessel wall to allow for the flow of small molecules (such as ions, water, and nutrients), large molecules (such as albumin, antibodies, cytokines, nucleic acids, and lipids), or even whole cells (such as lymphocytes, B cells, neutrophils, mast cells, macrophages, monocytes, eosinophils, and basophils) in to and out of the blood vessel.

The term “cytokine storm” as used herein means a severe form of systemic inflammatory response that arises as a complication of some stressor. A cytokine storm (also called hypercytokinemia) results in the release of more than 150 known inflammatory mediators (cytokines, oxygen free radicals, and coagulation factors). (Tisoncik, J. R. et al. Into the Eye of the Cytokine Storm. Microbiol. Mol. Biol. Rev. 2012 March, 76(1):16-32.)

The term “severe systemic inflammatory response” as used herein means any disease or condition in which a cytokine storm is induced.

As used herein, the term “sepsis” means a potentially life-threatening condition that occurs when the body's over reaction to an infection damages its own tissues, causing organs to function poorly and abnormally and even fail. Signs of sepsis include change in mental status, a drop in systolic blood pressure less than or equal to 100 millimeters of mercury (mm Hg) and a respiratory rate higher than or equal to 22 breaths a minute.

As used herein, “Coronavirus” or “Coronaviruses (CoV)” are a large family of viruses that cause illnesses ranging from the common cold to more severe diseases such as Middle East Respiratory Syndrome (MERS-CoV) and Severe Acute Respiratory Syndrome (SARS-CoV). SARS-CoV-2 is a new strain that was discovered in 2019 and has not been previously identified in humans. This virus causes COVID-19.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein the term “standard animal model” refers to any art-accepted animal model in which the compositions of the invention exhibit efficacy.

As used herein the term “post-acute COVID-19 syndrome (PACS)” refers to a condition in patients that have recovered from an initial COVID-19 infection but who are experiencing lingering sequelae, including anosmia, ageusia, shortness of breath, fatigue, cognitive issues, including memory loss and brain fog, erratic heartbeat, gastrointestinal issues, low-grade fever, intolerance to physical or mental activity, and muscle and joint pains. Patients with PACS are often referred to as “long-haulers” or “long COVID” patients.

### DETAILED DESCRIPTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the specific methods and materials are now described.

It must be noted that as used herein and in the appended claims, the singular forms “a,” “and” and “the” include plural references unless the context clearly dictates otherwise.

As described above, despite the fact that ST266 has been shown to have anti-inflammatory properties when delivered topically or locally, Applicant has demonstrated for the first time that systemically delivered ST266 is able to maintain its anti-inflammatory properties even when it is substantially diluted by blood. This is despite the fact that the cytokines and other therapeutic components in ST266 are at such low (physiologic) concentrations and they are immediately further diluted by the blood. For example, the average male has 5000-6000 mL of blood, so 35 mL of ST266 into that volume is ˜1:140 dilution.

Therapeutic Applications

Applicant believes that systemically delivered ST266 represents a novel therapeutic to treat the severe systemic inflammatory response termed cytokine storm.

Coronaviruses (CoV) are a large family of viruses named for the crown-like spikes on their surface. There are six known human coronavirus that cause illness ranging from the common cold to more severe diseases such as Middle East Respiratory Syndrome (MERS-CoV) and Severe Acute Respiratory Syndrome (SARS-CoV). The coronavirus causing the COVID-19 pandemic is termed SAR-CoV-2. It is a new strain that was discovered in 2019 and has not been previously identified in humans. Coronaviruses are zoonotic, meaning they are transmitted between animals and people. Detailed investigations found that SARS-CoV was transmitted from civet cats to humans and MERS-CoV from dromedary camels to humans. It is not yet known what animal transmitted SAR-CoV-2 to humans. Several known coronaviruses are circulating in animals that have not yet infected humans. Like other viruses, coronaviruses are known to mutate and several variants of SARS-CoV-2 have been identified around the world.

Common signs of infection include respiratory symptoms, fever, cough, shortness of breath and breathing difficulties and loss of smell (anosmia) and taste (ageusia). In more severe cases, infection can cause pneumonia, severe acute respiratory syndrome (SARS), kidney failure, cardiovascular problems and even death.

The release of numerous inflammatory cytokines plays significant roles in the severe clinical sequelae in patients with COVID-19. Highly pathogenic coronaviruses predominantly infect lower airways and can cause fatal pneumonia (Channappanavar, R. & Perlman, Seminars in Immunopathology (2017). doi:10.1007/s00281-017-0629-x). The body's response to severe infection in COVID-19 patients, particularly vulnerable patients such as the elderly and people with underlying medical conditions, is often a dangerous inflammatory overreaction known as a cytokine storm. The term was popularized during the avian H5N1 influenza virus infection (Tisoncik, J. R. et al. Microbiol. Mol. Biol. Rev. (2012). doi:10.1128/mmbr.05015-11). The COVID-19-infected population in Wuhan, China, where this new coronavirus was first identified, showed that plasma concentrations of IL-1β, IL-IRA, IL-7, IL-8, IL-9, IL-10, basic FGF, GCSF, GMCSF, IFNγ, IP10, MCP1, MIP1A, MIP1B, PDGF, TNFα, and VEGF concentrations were higher in both intensive care unit (ICU) patients and non-ICU patients compared to healthy adults (Huang, C. et al. Lancet (2020). doi:10.1016/S0140-6736(20)30183-5).

Acute lung injury (ALI) is a common consequence of a cytokine storm in the lung alveolar environment and systemic circulation and is most commonly associated with suspected or proven infections in the lungs or other organs (Rubenfeld, G. D. et al. N. Engl. J. Med. (2005). doi:10.1056/NEJMoa050333). In humans, ALI is characterized by an acute mononuclear/neutrophilic inflammatory response followed by a chronic fibroproliferative phase (McDermott, J. E. et al. BMC Syst. Biol. (2011). doi:10.1186/1752-0509-5-190). Thus, controlling the cytokine storm following COVID-19 infection is critical to preventing fatalities, especially in the elderly and those with chronic diseases such as diabetes, cancer, acute respiratory distress syndrome, hypertension and cardiovascular disease (Huang, C. et al. Lancet (2020). doi:10.1016/S0140-6736(20)30183-5).

Allogeneic mesenchymal stem cells (MSCs) were shown to significantly attenuate or significantly improve functional outcomes in seven treated patients with severe COVID-19 pneumonia (Leng, Z. et al. ChinaXiv (2020). Using MSCs to attenuate the cytokine storm associated with COVID-19 comes with numerous major challenges commonly associated with cell therapy. Tumorigenicity is an important risk factor (Neri, S. International journal of molecular sciences (2019). doi:10.3390/ijms20102406). Repeated administration of MSCs could result in allo-antibody production. Fetal bovine serum used in the MSC culture medium may elicit an immune response in patients. Delivery of MSCs also risks blood vessel occlusion. Widespread supply of MSCs that require expensive supply chain features such as ultralow cold storage to treat a worldwide pandemic present a major logistical distribution challenge (Musial-Wysocka, A., et al. Cell Transplantation (2019). doi:10.1177/0963689719837897).

Depending on the severity, other diseases or conditions may be classified as Systemic Inflammatory Response Syndrome (SIRS), most of which are characterized by a cytokine storm. These include sepsis, and Kawasaki's disease, as well as the newly identified COVID-19-associated syndrome resembling Kawasaki's known as Multisystem Inflammatory Syndrome in Children (MIS-C). Kawasaki's disease is an inflammatory condition that primarily affects children and is characterized by swelling in the walls of arteries, as well as lymph nodes, skin and mucous membranes inside the mouth, nose and throat. MIS-C, which has been associated with COVID-19 infection, is a similar condition wherein different body parts can become inflamed, including the heart, lungs, kidneys, brain, skin, eyes, or gastrointestinal organs. Several other inflammatory conditions, including polytrauma (i.e., having multiple traumatic injuries) idiopathic pulmonary fibrosis, graft-versus-host disease, multiple sclerosis, pancreatitis, irritable bowel syndrome and Crohn's disease have also been shown to exhibit similar features. Some autoimmune patients end up with cytokine storms (unrelated to COVID-19). This is most apt to occur in children with juvenile idiopathic arthritis (JIA). Adults with lupus, Still disease, and other inflammatory/autoimmune conditions may also develop a cytokine storm and therefore be treatable by ST266. In addition, although chimeric antigen receptor (CAR)-T cell therapy has experienced great success in the treatment of hematologic malignancies and is also promising for the treatment of solid tumors, it is associated with high rates of cytokine storm. (Garcia Borega, J., et al. HemaSphere: April 2019-3(2)). Thus, ST266 is suitable to help reduce the incidence of cytokine storm in patients undergoing CAR-T cell therapy.

In addition to the acute symptoms associated with COVID-19 infection, patients that have recovered from COVID-19 infections, regardless of whether or not they experienced acute symptoms, have reported lingering, long-term, weeks-to-months long symptoms. These include anosmia, ageusia, shortness of breath, fatigue, cognitive issues such as memory loss and brain fog, erratic heartbeat, gastrointestinal issues, low-grade fever, intolerance to physical or mental activity, and muscle and joint pains. These patients are experiencing what is generally referred to as post-acute COVID-19 syndrome, also commonly referred to as “long-haulers” or “long COVID”. Recent studies indicate that long-term neurological complications are affecting a significant percentage of post-acute COVID-19 patients (Aghagoli, et al. Neurocrit Care. 2020 Jul. 13: 1-10). The virus is thought to enter the nervous system through olfactory (sense of smell) neurons. Thus, a first sign of neurological injury or damage maybe anosmia (loss of smell). Viruses that enter the nervous system via the olfactory neurons may proceed to invade additional neural tissues.

Compositions and Methods of Making Compositions

Detailed information and methods on the preparation of AMP cell compositions, generation of ST266 (formerly ACCS) can be found in U.S. Pat. Nos. 8,058,066, 8,088,732, 8,278,095 all of which are incorporated herein by reference. Briefly, ST266 is made by obtaining a placenta and isolating an amnion from the placenta, enzymatically releasing amnion-derived epithelial cells from the amnion, collecting the released amnion-derived epithelial cells, culturing the collected amnion-derived epithelial cells in basal culture medium that is supplemented with 0.5% human serum albumin and 5-10 ng/mL recombinant human EGF, removing the culture medium after about 2-3 days and applying fresh culture medium, and collecting the culture medium after culturing the cells for 2-3 days.

The invention provides for an article of manufacture comprising packaging material and a pharmaceutical composition of the invention contained within the packaging material, wherein the pharmaceutical composition comprises ST266. The packaging material comprises a label or package insert which indicates that the ST266 contained therein can be used for therapeutic applications such as, for example, treating severe systemic inflammatory responses such as cytokine storm.

Formulation, Dosage and Administration of ST266 Compositions

Compositions comprising ST266 may be administered to a subject to treat severe systemic inflammatory responses such as cytokine storm.

Such compositions may be formulated in any conventional manner using one or more physiologically acceptable carriers optionally comprising excipients and auxiliaries. Proper formulation is dependent upon the route of administration chosen. For topical administration, the ST266 may be formulated as a spray, liquid, cream, foam, gel, lotion, salve, powder and ointment, etc. The compositions may also be administered to the recipient in one or more physiologically acceptable carriers. Carriers for ST266 may include, but are not limited to, solutions of normal saline, phosphate buffered saline (PBS), lactated Ringer's solution containing a mixture of salts in physiologic concentrations, or cell culture medium.

For parenteral administration, the formulation may be injected intravenously, although other routes of parenteral administration are contemplated by the instant invention, for example, intraperitoneal administration.

For enteral administration, the formulation may be administered as a liquid, a capsule or tablet that can be administered orally, rectally or gastrically.

For subcutaneous or intramuscular administration, the formulation may be delivered by needle and syringe, by pen injectors, by needle-less injection devices, and the like.

For nasal administration, the formulation may be administered as a nasal spray, a nebulized pulmonary dosage form, a metered dose inhaler or a dry powder inhaler.

For targeted intranasal administration the formulation is administered using a targeted delivery device. ST266 may be delivered using a number of currently available devices designed for intranasal nose-to-brain delivery. In one such embodiment, the device, or variants thereof, that is used is one described in, for example, U.S. Pat. Nos. 9,550,036, 10,507,295, 9,227,031, 9,339,617, 9,682,205, 10,099,019 or 10,549,052. SipNose LLC, located at 13 Hayetzira St., Yokneam Israel, 2066720 or online at sipnose.com, provides for suitable targeted intranasal devices.

In addition, one of skill in the art may readily determine the appropriate dose of ST266 for a particular purpose.

For ST266, exemplary intravenous doses may range from about 0.01 mL/kg to 100 mL/kg. In another embodiment and based on the studies set forth in Examples 4-7, a suitable range of ST266 is 0.5 to 1.0 mL/kg once or twice a day. One of skill in the art will recognize that the number of doses to be administered needs also to be empirically determined based on, for example, severity and type of disease, disorder or injury being treated; patient age, weight, sex, health; other medications and treatments being administered to the patient; and the like. For example, in a specific embodiment, one dose is sufficient to have a therapeutic effect (i.e., treating severe systemic inflammatory response such as cytokine storm). Other specific embodiments contemplate, 2, 3, 4, or more doses for therapeutic effect. Therapeutic effect may be measured by improvement in pulse oximetry, reduction of fever, or improvement or absence of worsening in clinical status on the WHO-7 point ordinal scale and compared to baseline (The Lancet 20(8):E192-E197 (2020). In addition, secondary endpoints may include change in TNFα, IL-1β or IL-6 levels from baseline.

The suitable dosages of ST266 are calculated from safety and proof-of-concept studies based on both animal pharmacodynamic and toxicology analyses. ST266 showed no toxicity in either rodent or large, non-rodent species at doses of up to 10 mL/kg body weight. The highest ST266 dose No Observed Adverse Effect Level (NOAEL) tested in beagle dogs was 5.0 mL/kg body weight. The suitable dosages are well below this level.

When delivered using targeted intranasal delivery, ST266 is delivered at a dose of 100 μL to 1 mL. In another embodiment, it is delivered intranasally at a dose of 200 to 400 μL daily. It may be delivered in one or both nostrils. Therapeutic effect may be measured by diminishment of anosmia from baseline using methods such as those described in World J Otorhinolaryngol Head Neck Surg. 2015 September; 1(1): 28-33 that describes The University of Pennsylvania Smell Identification Test (UPSIT) and the Olfactory Threshold Testing: The Single Staircase Odor Detection Threshold Test. In addition, measurement of viral-induced brain inflammation as measured by inflammatory markers in cerebrospinal fluid (CFS), such as those described in Cancer Cell 39(2):276-283 2021. Further, as the long-term effects of COVID-19 infection resemble those observed with Chronic Fatigue Syndrome, other measurements of efficacy may include improvement in Quality of Life, such as those described in Patient Relat Outcome Meas. 2018; 9:253-262.

One of skill in the art will also recognize that number of doses (dosing regimen) to be administered needs also to be empirically determined based on, for example, severity of the inflammatory response being treated; patient age, weight, sex, health, co-morbidities; other medications and treatments being administered to the patient; and the like. In addition, one of skill in the art recognizes that the frequency of dosing needs to be empirically determined based on similar criteria. In certain embodiments, one dose is administered every day for a given number of days (i.e., once a day for 7 days, etc.). In other embodiments, multiple doses may be administered in one day (i.e., every 4 hours, etc.). Multiple doses per day for multiple days (i.e., 2 doses a day for 7 days) are also contemplated by the invention.

To treat a severe systemic inflammatory response such as cytokine storm, the administration is intravenous. The timing for administration needs to be empirically determined by the attending physician as each patient will develop symptoms at different time points following onset. Optimally, ST266 will be administered as soon as symptoms first appear. This is necessary to prevent or minimize the excessive release of cytokines that occurs with cytokine storm, which could eventually cause death.

In further embodiments of the present invention, at least one additional agent may be combined with ST266. Such agents may act synergistically with the ST266 to enhance the therapeutic effect. Such agents include, but are not limited to, growth factors, cytokines, chemokines, antibodies, antibody cocktails, inhibitors, antibiotics, immunosuppressive agents, steroids, anti-fungals, anti-virals or other cell types. Inactive agents include carriers, diluents, stabilizers, gelling agents, delivery vehicles, ECMs (natural and synthetic), scaffolds, matrices and the like. When ST266 is administered conjointly with other pharmaceutically active agents, even less of the compositions may be needed to be therapeutically effective.

### Examples

The following examples are put forth so as to provide those of ordinary skill in the art with a complete disclosure and description of how to make and use the compositions and methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celsius, and pressure is at or near atmospheric.

### Example 1: ST266 in an Animal Model of Polytrauma

Polytrauma comprises two or more injuries with at least one injury being life-threatening. As described above, ST266 is a unique secretome obtained by culturing a novel population of cells termed Amnion-derived Multipotent Progenitor cells under proprietary, pharmaceutical grade cGMP conditions (D. Steed et al., Eplasty. 8, 157-165 (2008). ST266 contains hundreds of large molecular weight cytokines and growth factors in physiological concentrations that are involved in wound healing, neuroprotection, anti-inflammation, apoptosis prevention and vascular permeability reduction.

Method: The polytrauma model consists of a soft tissue and bone injury with rapid blood loss in a murine model (J. A. Luciano et al., Shock (2015), doi:10.1097/SHK.0000000000000412): (a) A soft tissue injury is performed using an 18-cm curved hemostat crushed with 270 psi on the bilateral lower extremities. (b) Previously prepared bone solution is injected bilaterally into the posterior muscles of each thigh at a volume of 0.15 cc using a 20-gauge needle. (c) 30% of the total blood volume (based on 80 ml blood/kg body weight) is rapidly removed within 1 minute using a 1-cc syringe and a 30-gauge needle via closed cavity cardiac puncture. (d) A midline incision made under the xyphoid, exposes the right middle lobe of the liver, is crushed 4 times at 80 psi using a 12.5-cm curved hemostat. Control groups were IV administered 1 mL/kg bolus of Lactated Ringers (LR) and experimental animals were IV administered a bolus of 1 mL/kg ST266. The bolus was followed by IV administration of 3 times the total shed blood volume (tSBV) of LR over 30 minutes.

Results: These investigative studies demonstrated that ST266 has the potential to reduce systemic inflammation following hemorrhagic shock characteristic of polytrauma. Plasma concentrations of 3 inflammatory cytokines, IL-6, IL-10 and TNF-α were consistently reduced at 6 and 24 hours in ST266-treated rats following removal of approximately 50-65% of the blood volume plus soft tissue injury. Furthermore, the liver enzymes AST, ALT and LDH trended lower in ST266-treated rats compared to LR treated animals at 6 and 24 hours following the procedure. These data support ST266's anti-inflammatory, anti-apoptotic and vascular permeability reducing properties observed in numerous other studies. These preliminary positive results support conducting additional experiments with greater animal numbers to demonstrate statistical significance. Future studies will be aimed at showing the histological effects of ST266 on liver, lung and other organs.

### Example 2: ST266 in an Animal Model of Emphysema

Method: Emphysema was induced in 8-week old C57BL/6 mice (n=10) by intratracheal administration of porcine pancreatic elastase (0.01 IU/gram of body weight). In this model the animals develop lung damage similar to clinical COPD in 28 days after elastase injection. Twenty-four hours after IT elastase, some animals received ST266 (0.5 ml, intraperitoneal (IP) or vehicle (n=10) daily for 14 days. Twenty 28 days after elastase injection, animals were euthanized and lungs were collected for histopathology and molecular studies. A group of animals (n=10) that did not receive elastase, ST266, or vehicle served as control. Data were expressed as mean f SEM.

Results: Alveolar Destructive Index (ADI), a measure of the severity of lung parenchymal destruction, was significantly lower (less destruction) in ST266 group compared to the elastase-only and vehicle-treated groups (26.74%±3.628%, 58.59%±2.149% and 49.99%±7.21%, respectively; p=0.0015)

Mean Linear Intercept (MLI), another measure of parenchymal destruction, was significantly lower (less destruction) in ST266 group compared to elastase-only and vehicle groups (55.26 μm±1.335 μm (mean±SEM), 75.43 μm±2.529 μm and 64.84 μm±6.105 μm respectively; p<0.0001).

There is a statistically significant decrease in the lung glutathione peroxidase level (GPX; nmol/ml of lung tissue) in the ST266 group compared to elastase-only and vehicle groups. (129.8±3.837, 156.7±6.609 and 142.7±8.409 respectively; p=0.0411).

Among the inflammatory cytokines we measured in the lung tissue after 28 days, IL-1β showed a significant decrease in the ST266 group compare to elastase-only and vehicle groups. (4.388±0.7324 vs. 13.58±1.794 and 13.92±1.207 (pg/ml of lung tissue): respectively; p=0.001).

### Example 3: ST266 Attenuates Neointima Formation and Luminal Stenosis after Arterial Balloon Angioplasty in Rats

Significant clinical evidence has emerged linking SARS-CoV-2 infection with endothelial cell dysfunction, increased vascular permeability and systemic inflammation (Jin Y, Ji W, Yang H, Chen S, Zhang W, Duan G., Signal Transduct Target Ther. 2020 Dec. 24; 5(1):293. doi: 10.1038/s41392-020-00454-7. PMID: 33361764; PMCID: PMC7758411). The endothelial cell protective and anti-inflammatory effects ST266 described herein are supportive of intravenous ST266 treatment in COVID-19 patients. (ClinicalTrials.gov Identifier: NCT04720378).

Methods—

Post-angioplasty restenosis due to neointima formation has been attributed to the inflammatory response after acute endoluminal injury. ST266 has been shown to be anti-inflammatory and to promote wound healing. This study examined the therapeutic potential of ST266 in a rat arterial balloon angioplasty model.

Neointima hyperplasia was induced in the iliac artery of Sprague-Dawley male rats using a 2F Fogarty embolectomy catheter. After surgery, the ST266 treated animal groups received 0.1, 0.5 or 1 mL intravenous (IV) ST266 once a day. Twenty-eight days after the surgery, the iliac arteries were removed for histologic analysis. Re-endothelialization index was measured 10 days after balloon angioplasty.

Results—

One mL of ST266 significantly decreased Neointima/Neointima+Media ratio (N/NM) compared with the saline control (0.34±0.01 vs 0.54±0.04, respectively; p=0.00⁴). ST266 also decreased Luminal Stenosis (LS) percentage compared with the control (18.18±1.86% vs 39.23±5.75%, respectively; p=0.008). One mL of ST266 significantly increased the re-endothelialization index 10 days after balloon angioplasty compared with the saline control treatment (0.40±0.04 vs 0.14±0.037, respectively, p=0.002).

Conclusion—

ST266 reduces neointima formation and luminal stenosis and increases the re-endothelialization index after balloon angioplasty.

### Example 4: ST266 Modulates Vascular Permeability In Vivo as Tested in the Miles Assay

Objective:

The purpose of this study was to evaluate whether or not ST266 can modulate vascular permeability in vivo using the Miles Assay (A. A. Miles AND E. M. Miles, Vascular reactions to histamine, histamine-liberator and leukotaxine in the skin of guinea-pigs, J. Physiol. (1952) 118, 228-257).

Method:

Evans Blue Dye (5%) was administered intravenously to male rats weighing approximately 300 grams. The Evans Blue dye binds albumin present in the animal's blood stream. Test groups with and without vascular permeability stimulants were then injected intradermally on the flank of the animal forming a small bleb. Changes in vascular permeability were measured by quantifying the amount of Evans Blue dye present in a skin biopsy taken from each bleb site. After dye was extracted from the skin biopsies, the sample absorbance at 630 nm (the Evans Blue peak wavelength) was normalized to the initial biopsy weight in grams. The vascular permeability stimulants tested in this manner included histamine, TNF-α, VEGF and bradykinin. Doses of stimulant were chosen based on literature references.

Results:

Table 1 below shows that compared to saline, ST266 reduced the Evans Blue signal induced by all stimulants tested. The reduction of Evans Blue is directly correlated with a reduction in vascular permeability at the injection site. Reduction in vascular permeability across all stimulants suggested that ST266 could be effective in multiple indications involving vascular permeability.

TABLE 1
Evans Blue dye Extraction (ABS₆₃₀/g tissue biopsy)

| 20 μg/mL | | 2 μg/mL | | 4 μg/mL | | 20 μg/mL | |
|---|---|---|---|---|---|---|---|
| Histamine | | TNF-α | | VEGF | | Bradykinin | |
| Saline | ST266 | Saline | ST266 | Saline | ST266 | Saline | ST266 |
| 1.330 | 0.291 | 0.505 | 0.203 | 1.449 | 0.519 | 1.28 | 0.71 |

### Example 5: ST266 Mitigation of LPS-Induced Inflammation in a Mouse Model of Cytokine Storm

Methods: This study demonstrates that a single dose of ST266 can significantly reduce inflammation in an established murine animal model of cytokine storm, the lipopolysaccharide (LPS) induced systemic inflammation model. In this model, (8-10) week old C57Bl6 male mice, body weight (BW) 25 g, were injected intraperitoneally (IP) with lipopolysaccharides (LPS.) In three treatment groups. Mice were injected into the opposite flank with either ST266 (IP) at 1 mL/Kg (low dose) or 40 mL/Kg (high dose) body weight (BW), 5 minutes after injection of LPS. Control animals were injected with an equal volume of 1×PBS in the opposite flank. Animals were bled and sacrificed at 2, 4 and 6-hours following injection. Blood samples were centrifuged, and the serum was collected and frozen for analysis of inflammatory cytokine levels. Serum concentrations of inflammatory cytokines were measured using the Meso Discovery (MSD) 200401 Murine Proinflammatory Antibody Array Panel for IFN-γ, IL-1β, IL-2, IL-4, IL-5, IL-6, KC/GRO, IL-10, IL-12p70 and TNF-α on a Meso Discovery, MESO QuickPlex SQ 120. Statistical significance of PBS placebo control groups and ST266 treatment groups were compared at various time points using Student t-test.

Results: LPS delivered one time at 5 mg/Kg BW was effective in inducing significant inflammation in the mice. Inflammatory cytokines measured using Meso Discovery Murine Proinflammatory Panel revealed that treatment with low dose ST266 at 1 mL/Kg resulted in almost all of the inflammatory cytokines showing reduction compared to the PBS control group at the 2 hr. time point. The inflammatory cytokine IL-10 was reduced by almost 30% compared to the PBS treated group. The inflammatory cytokines IL-2, IL-4 and IL-5 were reduced by 20.5%, 17.3% and 19.5%, respectively, in the ST266-treated animals compared to PBS controls. IL-10 showed a significant reduction at 2 hrs. (p<0.05). By 4 hrs., there were no notable differences between groups.

Following LPS injection (5 mg/Kg BW) and treatment with high dose ST266 IP (40 mL/Kg BW), 2 of the 10 inflammatory cytokines, IL-10 (p=0.018) and TNF-α (p=0.045) were significantly reduced 2 hrs. following injection with ST266 compared to PBS control. Additionally, ST266 resulted in 8 cytokines being significantly lower than PBS controls at 6 hrs. Individual p-values were: IFN-γ p=0.007, IL-1β p=0.027, IL-2 p<0.0001, IL-4 p=0.0164, IL-5 p=0.0295, IL-6 p=0.0171, KC/GRO not significant, IL-10 not significant, IL-12p70 p=0.0030 and TNF-α p=0.0058.

These data demonstrate that ST266 delivered systemically by IP injection reduces circulating inflammatory cytokine levels in an animal model of cytokine storm. In addition, mice injected with LPS and treated with ST266 had a 50% survival rate over LPS and placebo-treated mice.

### Example 6: Preclinical Safety/Toxicology in Rats

Preclinical safety/toxicology studies of intravenous ST266 were conducted in Sprague Dawley male and female rats. A 10 mL/kg BW dose was administered for 28 consecutive days and no adverse effects were observed.

### Example 7: 14-Day Repeat Dose Safety Study of Intravenously Administered ST266 in Male and Female Beagle Dogs

Method: A study was conducted to evaluate the general safety of ST266 during 14 consecutive days of once daily intravenous administration in male and female beagle dogs.

Daily mortality, clinical observations, body weight, and food consumption were monitored. Electrocardiograms were conducted on all dogs prior to the first dose and on Day 14 (following the last dose).

Results. No remarkable differences were observed by daily clinical observations, body weight or food consumption. There was no mortality among any of the animals in any dosing group. No significant changes in hematology, clinical chemistry, coagulation, or urinalysis were observed.

Based upon the observed results, 14 days of intravenous administration of ST266 at 0.5 mL/kg, 1.0 mL/kg, 2.75 mL/kg and 5.0 mL/kg body weight in male and female Beagle dogs was determined to be safe with no drug related adverse reactions.

Accordingly, these data support the dosing, including human dosing, based upon body weight normalization. Thus, in accordance with the invention disclosed herein, dosing of ST266 via the intravenous route is demonstrated at a dose of 0.5 mL/kg BW BID (twice a day) and escalating to 1.0 mL/kg BW QD (once a day).

### Example 8: ST266-Induced Immunogenicity Measured by Circulating Inflammatory Cytokine Concentrations in Beagle Dogs

Method: A study was conducted to confirm that ST266 is non-immunogenic as determined by measurement of circulating serum inflammatory cytokines following 14 consecutive days of once daily intravenous administration of ST266 in Beagle dogs.

Results: The concentrations of seven (7) out of eight (8) measured cytokines showed no significant difference between Day 1 and Day 15 (all with p>0.05, NS=Not Significant) for the entire data set of dogs. The data for the TNF-α cytokine is not reported as all serum measurements were below the lower limit of detection at both Day 1 and Day 15. These results confirm the absence of immunogenicity of extended administration of intravenous ST266 at levels of from 0.5 mL/kg BW/BID to 2.75 mL/kg BW/BID.

### Example 9: ST266 Promotes Survival and Maintenance of Core Body Temperature in a Mouse Model of Inflammation

Methods: Adult male C57BL/6N mice (12-weeks-old, body weight 25-30 g) were injected intraperitoneally (IP) with lipopolysaccharides (LPS); 15-20 mg/kg body weight, dissolved in 0.1 mL/10 g PBS (N=5/group) to induce inflammation and then subsequently administered an intraperitoneal (i.p.) injection of ST266 (30-40 mL/kg). Study endpoints included mortality and core body temperature assessments at 96 hours post-treatment.

Results: Treatment with a ST266 dose IP (33-40 mL/kg BW), showed a significant increase in survival with ST266 with 0 deaths at 96 hours vs 100% mortality for the LPS controls. ST266 also maintained core body temperature of the 15 mg/kg dosed LPS mice throughout the experiment. No adverse events were noted in the ST266-treated animals.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is:

1. A method for treating a severe systemic inflammatory response in a patient in need thereof comprising the step of systemically administering to the patient a therapeutically effective dose of ST266.

2. The method of claim 1 wherein the severe systemic inflammatory response is induced by an infectious or non-infectious stressor.

3. The method of claim 2 wherein the infectious stressor is selected from the group consisting of bacteria, viruses, and yeasts.

4. The method of claim 3 wherein the virus is a coronavirus.

5. The method of claim 4 wherein the coronavirus is SARS-CoV-2.

6. The method of claim 2 wherein the non-infectious stressor is selected from the groups consisting of surgery, trauma, autoimmune disease, burns, myocardial infarction, and chimeric antigen receptor (CAR)-T cell therapy.

7. The method of claim 1 wherein the systemic administration is intravenous administration.

8. The method of claim 1 wherein the therapeutically effective dose of ST266 is 0.01 mL/kg to 100 mL/kg.

9. The method of claim 8 wherein the therapeutically effective dose of ST266 is 0.1-1 mL/kg.

10. The method of claim 1 wherein the severe inflammatory response is Systemic Inflammatory Response Syndrome (SIRS).

11. The method of claim 1 wherein the severe inflammatory response is cytokine storm.

12. A method for treating cytokine storm induced by COVID-19 infection in a patient in need thereof comprising the step of administering to a patient a therapeutically effective dose of ST266, wherein the administration is intravenous administration and the therapeutically effective dose is 0.01 mL/kg to 100 mL/kg.

13. The method of claim 12 wherein the range of ST266 is 0.1-1.0 mL/kg once or twice a day.

14. A method for treating post-acute COVID-19 syndrome (PACS) in a patient in need thereof comprising the step of targeted administering to the patient of a therapeutically effective dose of ST266.

15. The method of claim 14 wherein the therapeutically effective dose of ST266 is 100 μL-1 mL/kg and is administered systemically.

16. The method of claim 14 wherein the therapeutically effective dose of ST266 is 200-400 μL daily and is administered intranasally.
